(19) United States
(12) Patent Application Publication
(10) Pub. No.: US 2020/0381109 A1
(43) Pub. Date: Dec. 3, 2020

(54) SYSTEM AND METHOD FOR EFFICIENT EQUIPMENT RESOURCE ALLOCATION

(71) Applicant: KONINKLIJKE PHILIPS N.V., Eindhoven (NL)

(72) Inventors: Márcia ALVES DE INDA, ROSMALEN (NL); Leonie Francelle WAANDERS, DRUTEN (NL); Wendy Uyen DITTMER, EINDHOVEN (NL); Nagaraju BUSSA, BANGALORE (IN); Arun Kumar MANI, MAYILADUTHURAI (IN); Janke Jörn DITTMER, EINDHOVEN (NL)

(21) Appl. No.: 16/995,184

(22) Filed: Aug. 17, 2020

Related U.S. Application Data
(63) Continuation of application No. 14/361,925, filed on May 30, 2014.

(30) Foreign Application Priority Data
Nov. 30, 2011 (IN) 4124/CHE/2011

(51) Int. Cl. G16H 40/20

(52) U.S. Cl. CPC G16H 40/20

(57) ABSTRACT

The present invention relates to systems for planning use of resources under consideration of time constraints. The present invention further relates to methods for scheduling use of resources under consideration of time constraints.

### CROSS REFERENCE TO RELATED APPLICATIONS

This application a Continuation of U.S. application Ser. No. 14/361,925 filed May 30, 2014, which is the U.S. National Phase application under 35 U.S.C. § 371 of International Application No PCT/IB2012/056545 filed Nov. 19, 2012, which claims the benefit of Indian Patent Application Number 4124/CHE/2011 filed Nov. 30, 2011. These applications are hereby incorporated by reference herein.

### FIELD OF THE INVENTION

The present invention relates to scheduling of a series of events, such as medical examinations for a patient in a clinical setting, such as a hospital or other medical examination set-up, in a psychological/educational evaluation, or any other situation where use of resources are to be scheduled to a number of users.

### BACKGROUND OF THE INVENTION

Cardiovascular disease (CVD) is one of the leading causes of death worldwide. Unlike other diseases, CVD can be prevented and often only with simple lifestyle modification measures. Although it is generally accepted that all individuals should aim at a healthy lifestyle in order to prevent CVD, there are individuals that are at higher risk (measured, e.g., by the Framingham 10 year risk) of experiencing a severe CVD event such as heart attack, stroke or death. It is thus desirable to effectively identify individuals who are vulnerable for a CVD event and refer them for medical treatment, lifestyle coaching, or other types of healthcare attention.

It is important to note that these higher risk individuals are generally persons in the population who are considered as healthy as they suffer from no symptoms. As a consequence it is preferred that determination of their risk should be easy, non-time consuming, and inexpensive.

Moreover as they feel healthy, motivating compliance with a treatment or a lifestyle change is an important aim. The results of the health status determination should be presented in a manner that the individual can understand and be motivated to comply with a clinician's recommendation. The patent and scientific literature demonstrates several methods for determining the risk score of an individual based on multiple parameters. These methods include calculating the Framingham, Reynolds, Rasmussen, SCORE, and PROCAM risks scores (see, for example, http://www.scopri.ch/riskalgorithms.htm). These risk scores are calculated by a clinician using software or tables. They provide an individual with a quantitative indication of her/his health status by indicating the likelihood of experiencing an acute event in the future. The calculated risks are used to determine whether an individual falls in the low, medium, or high risk category. Based on these categories certain treatment options can be recommended according to medical guidelines (e.g., Circulation 2004, 109, pages 672-693).

This simple model is often complicated by the fact that for the calculation of such risk scores several measurements on different types of instruments with varying complexity are required. Often individuals fall in an intermediate risk category and additional testing is required to reclassify them into a higher or lower risk category for determining follow up measures (e.g., prescribe medication vs. not prescribe medication). Moreover, frequently CVD is associated with other undiagnosed diseases (e.g., diabetes, hypertension, renal insufficiency, sleep apnea) which exacerbate the risk, and for which clinicians have to perform additional measurements and tests. These co-morbidities also need to be taken in account when developing check-up programs. In addition to the complex workflow the clinician also needs to account for the best practice guidelines and their updates.

Existing check-up programs are typically organized in three steps: an intake consult, followed by a battery of diagnostic tests, and a final consult where the results of the diagnostic tests are discussed and recommendations for follow-up actions (such as lifestyle modifications, extended diagnostic tests, treatment) are given. The different check-up models used can range from a “non-assisted check-up model”, where the consumer schedules the intake consult, diagnostic tests, and end consult by himself, without assistance (e.g., by making an appointment with his/hers general practitioner (GP), whom will request a number of tests, the consumer will then make the appointments for the diagnostic tests and, when the results are available, will schedule an new appointment with the GP to discuss the results), to check up packages that are offered by institutions where the consumer makes an appointment and all the necessary tests and consults are scheduled by the check-up service. In many cases the intake consult and tests take place at the same day but the end consult is scheduled for a different day. Or there is a preliminary end-consult and the consumer can choose to have a new consult when all results are ready, to have a telephone appointment, or simply receive the results at home. In other models the battery of tests in the check-up lasts more than one day and the consumer stays overnight in the check-up facility until all tests are completed and the end consult may occur on discharge, or there is a preliminary end-consult at discharge and the consumer can choose to have a new consult when all results are ready, to have a telephone appointment, or simply receive the results at home.

With the development of new point-of care diagnostic devices for which results of diagnostic tests can be obtained within an hour (or even minutes) it is possible to develop the concept of a “fast” check up package where the consumer has the whole check up package concentrated into a few hours without the need for different consults in different days or waiting for results that will be send by (e-)mail. In such a scenario, being able to schedule the intake consult, the battery of tests and the final consult in such way that the total check up time stays under a pre-determined time span becomes relevant. Especially in cases where the battery of tests offered by the check up package is highly personalized, with different tests being offered to different customers depending on their personal situation.
In such cases, the customary static scheduling solution which assumes that all the consumers will undergo the same series of tests in the same order (or even a more flexible scheduling solution that assumes a pre-defined reduced number of check up packages) is not suitable (this solution minimizes customer stay, but check up packages need to be pre-defined, and equipment may be idle unnecessarily). A more ‘equipment centric’ scheduling solution in where tests are scheduled separately for each diagnostic modality (i.e., the scheduler looks for an opening in the schedule of the specific diagnostic modality) is also not suitable, it does maximize equipment use, but customer does not minimize customer stay.

The inventor of the present invention has appreciated that an improved method and system for scheduling events, such as examinations for patients, is of benefit, and has in consequence devised the present invention.

### SUMMARY OF THE INVENTION

It would be advantageous to achieve a system that keeps the time spent on a series of related event, e.g. time spent by a person in a clinical setting such as a hospital or other examination set-up below a given threshold while maximising the utilisation of resources and number of recipients, i.e. persons. The resources include equipment, staff time, and the recipients could be a number of clients, e.g. patients or in general units, per day. In general, the invention preferably seeks to mitigate, alleviate or eliminate one or more of the above mentioned disadvantages singly or in any combination. In particular, it may be seen as an object of the present invention to provide a method that solves the above mentioned problems, or other problems, of the prior art.

Throughout the present description the term ‘patient’ is predominantly used, but other types of clients may be considered.

To better address one or more of these concerns, in a first aspect of the invention a system for scheduling a sequence of examinations for a patient in a hospital is presented that includes a first data base comprising data relating to a specific type of examination, the data including time consumption, and the data relating usage of resources associated with the specific type of examination, a second data base comprising data relating a patient to an associated list of examinations that the patient is to participate in, a scheduling unit operatively coupled to the first data base and the second data base, wherein the scheduling unit is arranged for scheduling the sequence of examinations for a group of patients based on their associated list of examinations and data from the first data base under consideration of a maximum total time used on a specific patient, the sequence including information relating to time and type of examination.

The system according to the first aspect may be used for scheduling tests for risk prediction of non-communicable diseases. A non-communicable disease is a medical condition or disease which is non-infectious. Non-communicable diseases are diseases of long duration and generally slow progression. They include heart disease, stroke, cancer, asthma, diabetes, chronic kidney disease, osteoporosis, Alzheimer's disease, cataracts, and more. Risk factors such as a person's background, lifestyle and environment are known to increase the likelihood of certain non-communicable diseases. They include age, sex, genetics, exposure to air pollution, and behaviour such as smoking, unhealthy diet and physical inactivity which can lead to hypertension and obesity, in turn leading to increased risk of many non-communicable diseases. Most non-communicable diseases are considered preventable because they are caused by modifiable risk factors.

Non-communicable diseases include many environmental diseases, covering a broad category of avoidable and unavoidable human health conditions caused by external factors, such as sunlight, food, pollution, and lifestyle choices. The diseases of affluence are non-infectious diseases with environmental causes. Examples include cardiovascular disease (CVD), chronic obstructive pulmonary disease (COPD), diabetes mellitus type 2, lower back pain caused by too little exercise and many more.

The system comprises information relating to a group of patients. The group of patients is preferably dynamic, so that when a new patient arrive the group, and conversely the schedule, is updated. Presently it is preferred that the group of patients are to be examined on the same day, and that the time constraint ensure that all patients are treated within a given maximum time interval. The present system may be used for planning a schedule for any day, e.g. the system may be used for recording that a patient e.g. calls and makes an appointment for another day, and an operator then enters this information to the system which than updates the collected schedule for that day for the patients currently schedule to be examined on that day.

The first data base comprises information on a number of examinations that the hospital is able to perform. Further, the first data base also includes statistical information on how long each type of examination usually consumes, this could include one or more of a minimum time, a mean time, a maximum time, a distribution for duration of the examination or operation. The data relating to usage of resources associated with the specific type of examination may include the number of examination stations, information on equipment, information on availability of operators, etc. The data in both the first data base and the second data base may be changed dynamically, e.g. new patients having a list of examinations, or an alteration in the list of examinations of a specific patient, and the schedule may then be accordingly updated.

Advantageously the system may include or receive information from a statistic data acquisition module. The statistic data acquisition module may collect or record data and prepare them in appropriate format for the system to perform optimal scheduling at any time.

When the scheduling unit determines the schedule, it takes into account at least the above-mentioned factors. The maximum time used may be set by an operator. The maximum time used may be individually set for each person in the group. The maximum time used may be set to a default value for persons having a specific medical condition or for all persons. The maximum time used may be re-set during the consultation. The maximum time used may be determined during a basic assessment at the start of the check-up, which means that the system may not know from the beginning what the maximum time for a patient actually is. Advantageously the system could at the beginning assign a default value for the expected maximum time used (e.g., the maximum of the maximum time, or the average maximum time) and refine this maximum time individually after the patient or client has passed though the basic assessment. Advantageously the system may warn the operator if it is not possible to obtain a schedule where all, or a given percentage of, maximum time used are fulfilled, and when to stop scheduling individuals for the day.

The scheduling unit may be constituted by a processor, e.g., in a computer device. The processor may then be in communication with data storage having the first and second data bases. The first and second data bases may be separate data bases or included in one data base and arranged so as to logically appear separate or as entries in one single data base.

Advantageously the first data base may further comprise dependency information for the specific types of examinations, and the scheduling unit may be arranged for scheduling the sequence of a number of examinations in view of these dependencies. When scheduling a series of examinations there may be certain dependencies to be taken into account, e.g., certain examinations may need to be performed first so that the result of that examination is available before another examination is to be performed, or two measurements may not be performed in parallel, or two measurements may be performed in parallel. For example, the same instrument could perform two measurements at the same time; e.g. blood (previously collected) is being tested while another examination is taking place.

Advantageously the first data base may further comprise information relating to estimated time use for information conveyance from a health care person to a patient for one or more of the specific type of examinations. As patients are subjected to a number of examinations, they may feel a need for being informed as to what the examination entails. This conveyance of information takes some time, which may depend on the complexity of the examination. Therefore this period of time may need to be included in the schedule as the equipment and/or personnel would otherwise delay the rest of the schedule. The data base may further comprise information on the average time is takes for a patient to move between two examination rooms. In a large hospital this time may be significant.

Advantageously the first data base may further comprise data relating to availability of clinicians and/or operators associated with a specific piece of equipment, and the availability of the equipment, e.g. a given piece of equipment may be serviced by a technician, or be non-functioning and awaiting such service. When determining a schedule for a patient there may be a need for considering if the equipment and/or personnel are available also. Further, there may be one number of stations/equipment available and another, possibly lower, number of personnel to operate the equipment available. The scheduler may then take the availability of these resources into account.

Advantageously the scheduling unit may include time constraints relating to maximum time used on a specific patient in scheduling the sequence of examinations. The maximum duration of total time may be up to most of a day, e.g., 10 hours, 9 hours, 8 hours, 7 hours, 6 hours, 5 hours, 4 hours, 3 hours, 2 hours or 1 hour or any other suitable maximum time period.

Advantageously the system may further comprise a transmitter for transmitting a schedule comprising a sequence of examinations to a patient receiver unit. The patient receiver unit is preferably a portable unit where the patient may retrieve information relating to the schedule of examinations. The information on the patient receiver unit may be updated with real-time information, e.g. time to next examination. The patient receiver unit may be supplied by the hospital and be a customized unit or be a mobile phone that the patient is already in possession of The patient receiver unit may include a software application identifying the patient, collecting/recording/receiving the schedule, displaying and/or playing sound and/or video, e.g., relating to an examination, or lifestyle modification advice.

Advantageously the patient receiver unit may further comprise information relating floor plan indicating to the patient where specific examinations occur. As hospitals are usually large buildings with many separate departments each conducing specialised tasks the patient may experience difficulty in finding the correct location for a scheduled examination. This may result in the patient being late for the next scheduled examination, and having a floor plan made available to the patient on the patient receiver unit provides the patient with the possibility to arrive at the correct location on, or before, the correct time, thus reducing idle time.

Advantageously the first data base may further comprise information relating to the specific examinations, the patient receiver unit arranged for displaying text and/or video and/or audio relating to an upcoming examination in the schedule. The conveyance of information to the patient relating to an upcoming examination may reduce the time spent by the health care personnel so that the time may be spent on performing the examination.

Advantageously the first data base may further comprise information relating to follow-up life-style coaching options, clinical follow up options, the patient receiver unit arranged for displaying text and/or video and/or audio relating to advisable follow-up options. The conveyance of information to the patient relating follow up may reduce the time of spent by the health care personnel so that the time may be spent on performing the final consult.

Advantageously the system may further comprise an input unit for inputting progress information on progress of patients and the system being arranged for adapting schedules for each of the patients in the patient group based on the progress information. The system may be arranged for adapting schedules for each of the patients in the patient group in response to the information on progress of patients. The system may determine that schedules are to be adapted based on progress information when it is determined that the current schedules are no longer possible or no longer acceptable. Further, the system may indicate to an operator that it is not possible to determine a schedule where the time constraints are complied with. The input unit may, e.g., be a unit at the place where a specific examination has taken place, the unit may, e.g., be a computer device operated by the health care person performing the examination inputting to the system that a specific patient has completed the examination or even an automated unit recording events, e.g., end of examination etc. The information may include time information, e.g., time of start, time of conclusion, duration of examination etc.

Advantageously the system may further comprise a patient presence indication unit for indicating to the system if a patient is present at the scheduled examination at the scheduled time. The patient presence indication unit may indicate the location of the patient, thereby allowing the system to evaluate if the patient is at the correct location and/or determine an expected time of arrival at the next scheduled examination. Further the system may suggest a route to the patient to the next location that he or she is scheduled to be at.

In an alternative wording the present invention may relate to a system for scheduling a sequence of events for a group, the system may comprise a first data base comprising data relating to a specific type of events, the data including time consumption, and the data relating usage of resources associated with the specific type of event, a second data base comprising data relating an individual or an object to an associated list of events that the individual or object is to participate in, a scheduling unit operatively coupled to the first data base and the second data base, wherein the scheduling unit is arranged for scheduling the sequence of events for a group of individuals based on their associated list of events and data from the first data base under consideration of a maximum total time used on a specific individual or object, the sequence including information relating to time and type of event.

This alternative may utilise or include any features mentioned in relation to the first and/or second aspect of the present invention.

A second aspect of the present invention relates to a method for scheduling a sequence of examinations for a group of patients using a system comprising: a first data base comprising data relating to a specific type of examination, the data including time consumption, and the data relating usage of resources associated with the specific type of examination, a second data base comprising data relating a patient to an associated list of examinations that the patient is to participate in, a scheduling unit operatively coupled to the first data base and the second data base, the sequence including information relating to time and type of examination, the method may then comprise the scheduling unit determining the sequence of examinations for each individual in the group of patients based on their associated list of examinations and data from the first data base and under consideration of a maximum total time used on a specific patient. The method allows a schedule to be determined for a group of patients. As with the first aspect the group may be dynamically determined, i.e. an operator may add or remove a patient from the group if he or she cancels or wish to be included in the schedule for a particular day. The method may include any features mentioned in relation to the system according to the first aspect.

The method according to the second aspect may advantageously be computer implemented and the steps may be performed using a processor.

Advantageously the first data base may further comprise dependency information for the specific types of examinations and the method may comprise the scheduling unit determining the sequence of examinations under consideration of the dependency of each examination. As the examinations may have some relative dependencies there is a need for considering them when scheduling a sequence of examinations.

Advantageously the method may further comprise an initial step of defining the associated list of examinations based on the specific patient's medical condition and/or on an initial assessment of the patient's condition. The associated list of examination may, e.g., be defined using a Clinical Decision Support System (CDSS), or simply be defined by, e.g., a physician. This “initial” step may be repeated during the check up and the examination list may be further modified as the results of previous examinations are ready and the CDSS (or physician) determine the need for more (or less) examinations. The associated list may be adapted using an input unit, e.g. a keyboard operated by a heath care person. The associated list may be adapted based on results from examinations performed.

Advantageously the system may comprise a transmitter for transmitting a schedule comprising a sequence of examinations to a patient receiver unit and the method may comprise a step of transmitting a schedule to a patient receiver unit associated with a specific patient. As discussed above the patient receiver unit is preferably a portable unit where the patient may retrieve information relating to the schedule of examinations. The information on the patient receiver unit may be updated with real-time information, e.g., time to next examination. The patient receiver unit may be supplied by the hospital and be a customized unit or be a mobile phone that the patient is already in possession of. The patient receiver unit may include a software application identifying the patient, collecting/recording/receiving the schedule, displaying and/or playing sound and/or video, e.g., relating to an examination, or lifestyle modification advice.

Advantageously the system may further comprise a real-time data acquisition module and the scheduling unit being arranged for adapting the scheduled sequence based on information from the real-time data acquisition module. The method may include updating or adapting schedules based on input relating to actual status of a patient, e.g., information relating to location, end of examination, delay in schedule from one or more examinations, change of schedule, and any other information relevant to the schedule. The list of examinations may be changed due to results obtained in a previous examination, e.g. the need for further examinations or cancellation of later examinations.

Advantageously the system may further comprise a time usage data acquisition module that accumulates data related to the usage of equipment (e.g., duration of use for each examination, down time for servicing purposes), the duration of each examination and the total time used for the check-up. The data collected by the time usage data acquisition module may then be statistically processed. The method may include updating, or adapting, the first data base with the data collected and/or processed. The statistical processing may include calculation of mean value or determining distribution and the like.

In general the various aspects of the invention may be combined and coupled in any way possible within the scope of the invention. These and other aspects, features and/or advantages of the invention will be apparent from and elucidated with reference to the embodiments described hereinafter.

### BRIEF DESCRIPTION OF THE DRAWINGS

Embodiments of the invention will be described, by way of example only, with reference to the drawings, in which:

FIG. 1 is a schematic illustration of a system having a first and a second data base,

FIG. 2 is a schematic illustration of a system having a data base combining the first and second data base,

FIG. 3 is a schematic illustration of a system having a transmitter

FIG. 4 is a schematic illustration of a personal receiver unit,

FIG. 5 schematically illustrates steps of a method for scheduling examinations,

FIG. 6 is a schematic illustration of steps of modifying a schedule, and

FIG. 7 schematically illustrates a check-up work flow.

### DESCRIPTION OF EMBODIMENTS

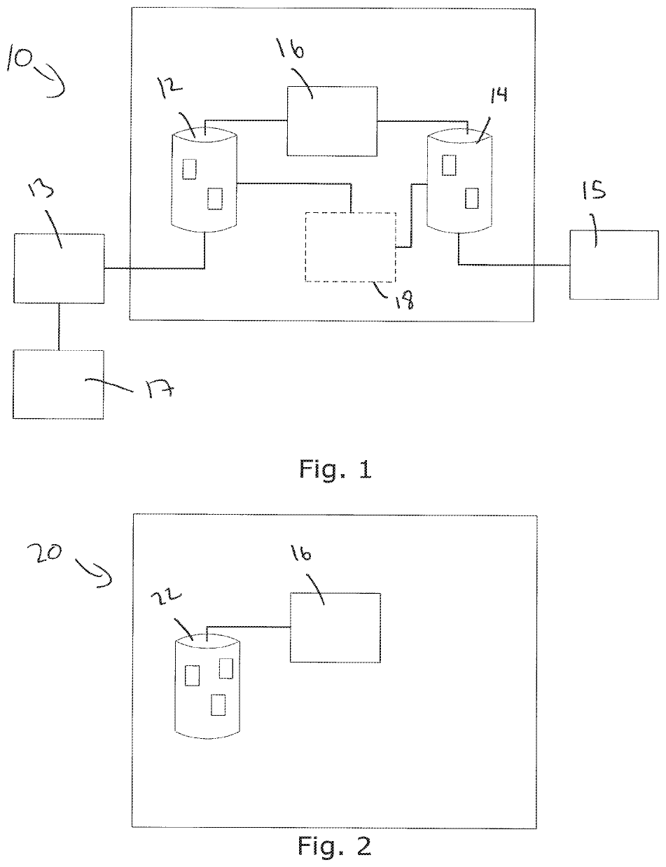

FIG. 1 schematically illustrates a system 10 for scheduling a sequence of examinations of a patient in a hospital. The system 10 is to be used in clinical environments, such as hospital settings, but may also be used in other environments, e.g., clinics, or health centre, or primary care centre, or other non-clinical evaluation center with similar scheduling needs. The system 10 comprises a first data base 12 comprising data relating a specific type of examination to statistical data relating to time consumption for the specific type of examination and data relating to usage of resources associated with the specific type of examination. This data base 12 comprises information that allows the system 10 to optimise the schedules with a certain probability of the schedule being complied with.

The system 10 further comprises a second data base 14. The second data base 14 comprises data relating to a patient and an associated list of examinations that the patient is to participate in and time constraints relating to maximum time used on a specific patient in scheduling the sequence of examinations. The time constraints may alternatively be stored in other storages. This data relates to the patient and the time constraints imposed in the scheduling. The time constraints, e.g., the maximum duration of total time used on a specific patient may be set by an operator. The maximum duration of total time used on a specific patient may also be set by hospital management and be a maximum value for, e.g., a group of patients or a specific department or type of examinations, e.g., cardio vascular examinations, individuals at intermediary risk of a cardiovascular event, diabetic individuals.

The system 10 further comprises a scheduling unit 16 operatively coupled to the first data base 12 and the second data base 14. The scheduling unit 16 may be coupled to the data bases 12, 14 via data paths. The scheduling unit 16 is in a presently preferred embodiment a computer processor with appropriate software. The scheduling unit 16 is arranged for scheduling the sequence of examinations for a group of patients based on their associated list of examinations and data from the first data base 12 and the second data base 14. The output from the scheduling unit 16, i.e., the sequence, includes information relating to time and type of examination. This allows the patient, or each of the patients, to be informed on what examination is to be performed when. By knowing this the patient is able to arrive at the examination without delay. This optimises the usage of the equipment as well as keeping the time spent in the hospital or clinic by the patient below a maximum value.

The system comprises a problem definition module 18 which is in charge of acquiring information including the check-up workflow, which includes possible pathways, and information relating to what tests have to be performed, is it necessary to include an intermediate consult to decide if more tests are necessary and the like. This information is preferably stored in the first data base. Also information relating to duration of tests (how long does it take on average, worst case), dependency graph for the tests (which test depend on other tests, which tests can be performed in parallel), and finally other aspects such as: relaxation time before or after a measurement, walking time to other test spaces, (un)dressing time, explanation time may be stored in the first data base. All of these data may be of help for determining a schedule for the one or more patients that will most likely be complied with. As the schedule may take into account the above mentioned factors, there is an increased likelihood that the patient will experience that the examinations occur at the scheduled times. The patient may receive the entire schedule at one time, or have presented a limited list of examinations, e.g., the next few examinations, and related times. The problem definition module 18 inputs the data to the first data base 12 and the second data base 14. The problem definition module 18 is indicated as the box with dashed lines in FIG. 1 as it is an optional module. The module 18 may also be included in the other embodiments illustrated in the present specification.

The system 10 may further have access to data relating to human and material resources to consider, including clinicians/operators (number, speciality), rooms (number, layout, equipment present), equipment (number, function), time constrains, e.g., maximal time per client, opening times, personnel work schedule, scheduling type: static (with appointment) or dynamic (without appointment). These data may be stored in the first data base 12. This data is preferably regularly updated based on data collected by the time usage acquisition module.

The scheduling unit 16 is in charge of determining the schedule based on the problem definition data and on the real-time data acquired. The scheduling unit 16 may employ any appropriate existing scheduling algorithm, e.g., linear programming, genetic algorithms, or any other suitable algorithm including numerical algorithms, or a combination of those to solve the problem with the goal of keeping the total patient stay shorter than the pre-determined maximum, while maximizing the number of patients that can be served in a day.

The system 10 is in communication with an input unit 15, e.g. a keyboard operated by an operator or a separate system for maintaining the data in the second data base 14. The input unit 15 may further be controlled by an external CDSS system.

A statistical process unit 13 collects data from a data acquisition module 17. The data acquisition module 17 collects various types of timing data from the examination stations. This allows the system 10 to update, in real-time, the schedules in case of delays, changes in patients examination lists etc. The data acquisition module 17 logs data from examinations, this data could be obtained in an automated fashion or inputted by an operator manually. The statistical process unit 13 may perform statistical analysis on the data, e.g. calculate average examination times, average delays etc. This data may be used (off line) to update the statistical data in the first data base.

FIG. 2 schematically illustrates a system 20 where the data from the system 10 are collected in one data base 22. Compared to the system 10 the data base 22 comprises the same type of data as the two data bases 14 and 12. As discussed elsewhere, the data in the data base 22 may be organised so as logically to appear as two separate data bases to the user.

Common for both the system 10 and the system 20, the data relating to the examinations, i.e., the data comprised in the first data base 14, may further comprise dependency information for the specific types of examinations. This information ensures that certain examinations that require results from other examinations are performed first. When this information is available to the scheduling unit 16, the scheduling unit 16 is arranged for scheduling the sequence of examinations in view of the dependencies. Thereby it is ensured that the examinations are performed in the correct order.

In some embodiments, the system 10, 10′, 20 may comprise information relating to estimated time use for information conveyance from a health care person to a patient for one or more of the specific type of examinations. As the patient may be subjected to the examinations for the first time there may be a need for a health care person, e.g., doctor, nurse, radiologist, laboratory technician or the like, to inform the patient what the examination entails. This takes some time and when statistical information relating to this process is available the scheduling unit may take this into account. The scheduling unit may have access to a record indicating if the is the first time the patient is part of the specific examination or not and may use the information to estimate how much time is spent on information conveyance. This may reduce unexpected delay in the overall schedule.

The system 10, 20 may be in communication with other planning systems, e.g., planning of clinicians work schedules, so that information on availability of persons and/or equipment is available to the scheduling unit 16.

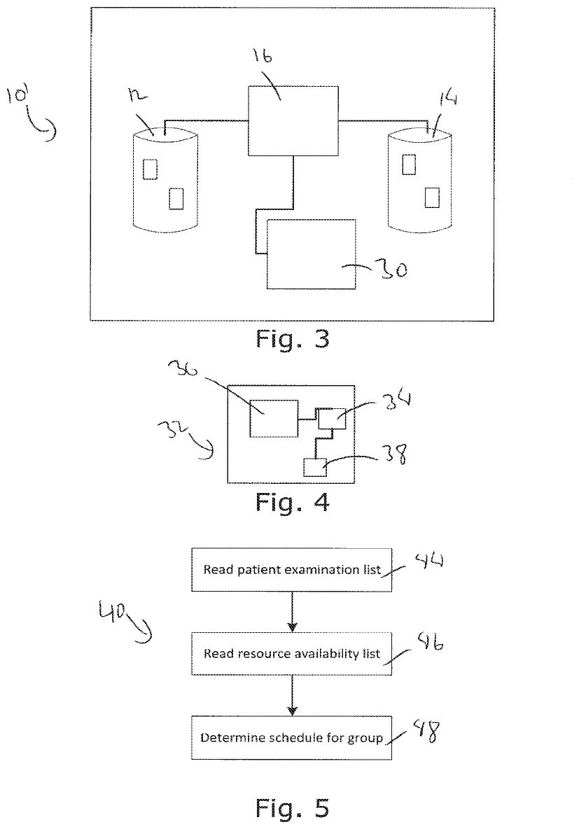

FIG. 3 schematically illustrates a system 10′, similar to that illustrated in FIG. 1, comprising a transmitter 30 for transmitting a schedule comprising a sequence of examinations to a patient receiver unit. The transmitter 30 may be a transceiver so that information from the patient receiver unit may be returned to the system.

FIG. 4 schematically illustrates a patient receiver unit 32. The patient receiver unit 32 comprises a processor 34 and a display unit 36. The receiver 38 receives data from the system 10, 20; the processor processes the data and displays the data on the display unit 36.

In an embodiment the patient receiver unit 32 further comprises information relating floor plan indicating to the patient where specific examinations occur. This allows, e.g., route information to be displayed the display unit 36 so as to help the patient to the correct location in time. Further, the patient receiver unit 32 may be arranged to display the current location of the patient along with the location of the next examination, or each examination, so as to allow the patient to gain a graphical overview of the building, or buildings, that the examinations are performed in. The patient receiver unit 32 may further comprise a location detection unit and the system 10, 20 may then be arranged for determining estimated time to travel from a current location to the location of the next examination and convey the estimated time to travel to the patient and back to the scheduling system. This allows the system to take into account the possibility of delays due to the patient turning up too late and/or conveying a message to the patient that he or she seems to be delayed in arriving at the next examination.

As mentioned, the examinations may be complex and/or unfamiliar to the patient. Therefore the patient may experience it as a help to be supplied with a portable unit where a screen may display information on the examination that he or she is going to be submitted to. Also the patient receiver unit 32 may include a speaker unit, or head set port for connecting a headset, so as to play sound recordings regarding the examination. The first data base may comprises information relating to the specific examinations and the patient receiver unit 32 may then be arranged for displaying text and/or video and/or audio relating to an upcoming examination in the schedule. This may done, e.g., while the patient is travelling between examinations or sitting in a waiting room, thereby reducing the time spent by the medical staff for conveying the information to the patient.

As also mentioned the first data base may further comprise information relating to follow-up life-style coaching options, clinical follow up options, and the patient receiver unit 32 may then be arranged for displaying text and/or video and/or audio relating to advisable follow-up options. The conveyance of information to the patient relating follow up may reduce the time of spent by the health care personnel so that the time may be spent on performing the final consult.

In an embodiment the system 10, 10′, 20 may further comprise an input unit for inputting information on progress of patients and the system being arranged for adapting schedules for each of the patients in the patient group based on the progress information. This will allow the actual progress to be recorded and the schedule to be checked for need of updating time and/or examination if delays cause significant changes in the collected schedule for the group of patients. The system may then be able to dynamically adapt the schedule if necessary, e.g., if a patient cancelled or added to the group, someone from staff is not available, a device is broken or other events changing the basis for the schedule.

The patient receiver unit 32 may be used as a real-time acquisition module to acquire real time information needed for the system, including patient presence and/or location. The system 10, 20 may further be arranged for receiving information regarding changes in medical staff availability, resources availability and other information that have an impact on the possibility to proceed according to the schedule.

The system 10, 10′, 20 is preferably connected to a schedule presentation module that is arranged for displaying the schedule information and/or is arranged to connect with a primary CDSS to transmit scheduling information and/or is arranged to connect with the patient receiver unit of each patient and to transmit scheduling information pertinent to that patient. This could be done via 15 as described above.

The patient receiver unit 32 may further comprise a patient presence indication unit for indicating to the system 10, 20 if a patient is present at the scheduled examination at the scheduled time. This will allow the system 10, 20 to update the schedule in case a patient is delayed in any way.

FIG. 5 schematically illustrates steps of a method 40 for scheduling a sequence of examinations for a group of patients using a system as discussed in relation to FIGS. 1-4. The method 40 comprises generally the step of the scheduling unit determining the sequence of examinations for each individual in the group of patients based on their associated list of examinations and data from the first data base 12 and the second data base 14. This means that the scheduling unit 16 will read or load a list of examinations for each patient of the group, step 44, the scheduling unit 16 will read or load a list for the availability for resources, step 46, and then determine a schedule for the group of patients, under consideration that the time spent for each patient is below a given threshold, step 48. The threshold may, as discussed above, be individual for each patient or a subgroup of patients. The schedule may be determined using linear programming methods or other suitable methods.

As mentioned in relation to FIG. 1 the first data base may comprise dependency information for the specific types of examinations and the method 40 may then comprise the additional step of the scheduling unit 16 determining the sequence of examinations under consideration of the dependency of each examination. As mentioned this ensures that the patient will undergo the examinations in view of any required order.

The method may comprise an initial step and intermediary steps of defining the associated list of examinations based on the specific patient medical condition, or risk group. This may, e.g., be performed in a CDSS system associated with the system 10, 10′, 20.

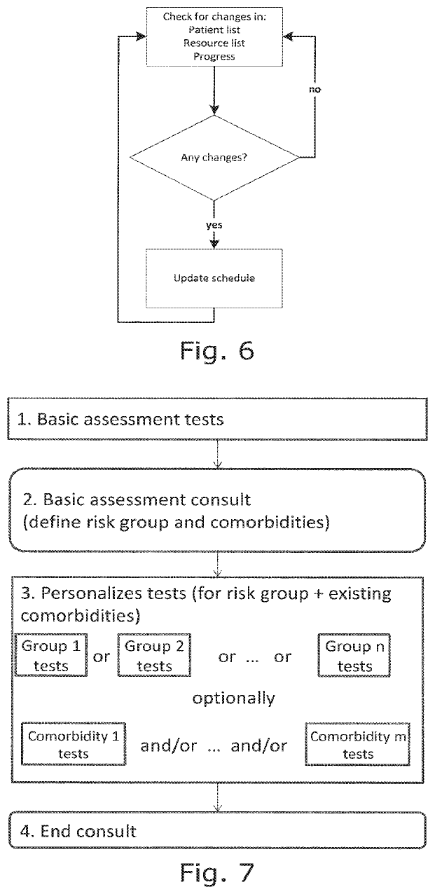

FIG. 6 schematically illustrates steps performed when monitoring the system if changes are made to the patient list, the resource list and/or timing issues. The patient list may be updated as patients arrive, leave early, are added to the group, or removed from the group, e.g. if they cancel or does not show up. The resources may be changed or updated as equipment breaks down, is to be serviced or other issues arise. The timing issues could include delays, e.g. patients do not finish an examination on time, or is delayed in traveling between examination stations, uses more time than expected in receiving information etc. As illustrated in FIG. 6 the monitoring may be performed continuously.

The method 40 may comprise a step of transmitting the schedule to a user, e.g. via the before mentioned patient receiver unit 32 or via an operator e.g. at an examination station where the operator then tells the patient where to go next or by other means.

The method may advantageously be implemented as a software method for use in a computer system. The resulting software may be stored on a data storage medium such as but not limited to DVD, CD-ROM, Blu-ray, hard drive, or flash memory device.

FIG. 7 schematically illustrates a flow diagram illustrating the steps of a check-up workflow that a patient goes through. All patients go through the basic assessment tests and basic assessment consult. During the basic assessment consult the patient is classified into a risk group (e.g., low risk, intermediary risk, high risk). Depending on the risk group and existence of relevant comorbidities a personalized list of tests is defined. Each risk group and comorbidity group has an associated list of tests.

The patient goes through the tests in the personalized list of tests. This personalized list may be modified in during the personalized test execution. Some may be added or dropped depending on the results of other tests.

The test results and follow-up recommendations are discussed in the end consult, preferably with a qualified health-care person.

This data would be used by the associated CDSS, and it is used to create/modify the patient examination list. The scheduling system itself does not need to know all details but may simply receive the information from the CDSS or via an input device as discussed above.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. A single processor or other unit may fulfil the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. Any reference signs in the claims should not be construed as limiting the scope.

1. A system comprising:
a controller;
a communication network;
a plurality of at least five equipment resources,
wherein each equipment resource has a corresponding equipment resource utilization time,
wherein the equipment resource utilization time is a time duration that the equipment resource incurs for each equipment resource allocation;
an equipment resource demand scheduler,
wherein the equipment resource demand scheduler determines a set of allocations of time segments for the plurality of equipment resources, based on each equipment resource's utilization time,
wherein the set of allocations of time are based on a plurality of demands for a plurality of at least five users,
wherein each plurality of demands includes a demand for allocation of at least three equipment resources;
wherein a demand duration for each user corresponds to a time duration between an earliest allocation of equipment resource to the user and a latest allocation of equipment resource to the user to satisfy the plurality of demands of the user;
wherein the controller communicates the allocations to the plurality of users via the communication network;
a monitoring system that reports a current utilization of each equipment resource to the controller;
wherein the controller compares the current utilization of each equipment resource to the allocation of time segments for each equipment resource to determine if a modification is required to the set of allocations of time segments;
wherein, if the modification is required, the controller instructs the equipment resource demand scheduler to provide the modification of the set of allocations, and provides the modified allocations to the plurality of users via the communications network;
wherein, the equipment resource demand scheduler performs the modification by iteratively:
identifying an alternative set of allocations;
determining the demand duration for each user with this alternative set of allocations;
determining if the demand duration for each user is within a pre-defined maximum demand duration for each user; and
repeating the identifying and determining steps until the demand duration of all users are below the maximum demand duration for each user.

2. The system of claim 1, wherein the equipment resources are medical equipment resources and the users are patients.

3. The system of claim 1, wherein the equipment resource demand scheduler allocates the time segments of the plurality of equipment resources based on sequence dependencies in the plurality of demands.

4. The system of claim 1, wherein the controller also communicates instructions regarding travel to each allocated equipment resource to each user via the communication network.

5. A method of allocating equipment resources comprising:
identifying a plurality of at least five equipment resources,
wherein each equipment resource has a corresponding equipment resource utilization time,
wherein the equipment resource utilization time is a time duration that the equipment resource incurs for each equipment resource allocation;
determining a set of allocations of time segments for the plurality of equipment resources, based on each equipment resource's utilization time,
wherein the set of allocations of time are based on a plurality of demands of a plurality of at least five users,
wherein each plurality of demands includes a demand for allocation of at least three equipment resources;
wherein a demand duration for each user corresponds to a time duration between an earliest allocation of equipment resources to the user and a latest allocation of equipment resources to the user to satisfy the plurality of demands of the user;
communicating the allocations to the plurality of users;
receiving a current utilization of each equipment resource;
comparing the current utilization of each equipment resource to the allocation of time segments for each equipment resource to determine if a modification is required to the set of allocations of time segments;
wherein, if the modification is required, providing the modification of the set of allocations, and providing the modified allocations to the plurality of users;
wherein, providing the modification includes:
identifying an alternative set of allocations;
determining the demand duration for each user with this alternative set of allocations;
determining if the demand duration for each user is within a pre-defined maximum demand duration for each user; and
repeating the identifying and determining steps until the demand duration of all users are below the maximum demand duration for each user.

6. The method of claim 5, wherein the equipment resources are medical equipment resources and the users are patients.

7. The method of claim 5, wherein the allocation of the time segments of the plurality of equipment resources is based on sequence dependencies in the plurality of demands.

8. The method of claim 5, including communicating instructions regarding travel to each allocated equipment resource to each user.
